(19) United States
(12) Patent Application Publication
(10) Pub. No.: US 2021/0308297 A1
(43) Pub. Date: Oct. 7, 2021

(54) AUTONOMOUS SANITIZATION MANAGEMENT, CONTROL AND DATA PLATFORM

(71) Applicant: Maxwellian Inc., Montreal (CA)

(72) Inventor: Alireza Hassani, Verdun (CA)

(21) Appl. No.: 17/151,878

(22) Filed: Jan. 19, 2021

Related U.S. Application Data
(60) Provisional application No. 63/076,414, filed on Sep. 10, 2020.
(60) Provisional application No. 63/019,555, filed on May 4, 2020.
(60) Provisional application No. 63/003,679, filed on Apr. 1, 2020.

(51) Int. Cl. A61L 2/10; A61L 2/24; A61L 2/26; B64C 39/02

(52) U.S. Cl. CPC A61L 2/10; A61L 2/24; A61L 2/26; B64C 2201/145; A61L 2202/11; A61L 2202/14; A61L 2202/16; B64C 39/024

(57) ABSTRACT

An autonomous sanitization management, control and data platform (ASMCDP) for sanitizing an area includes an autonomous sanitization control center (ASCC) and one or more standalone autonomous sanitization (SAS) units communicatively coupled to the ASCC. Each of the one or more SAS units includes at least one sensor, a microcontroller communicatively coupled to the at least one sensor, and a light source coupled to the microcontroller. Power to the light source is selectively controlled by the microcontroller in response to a triggering signal emitted by the at least one sensor. When the light source is powered on, the light source emits sanitizing radiation. The one or more SAS units may be mounted within a vehicle to enable sanitization of the vehicle passenger compartment.

### CROSS-REFERENCE TO RELATED APPLICATIONS

This application claims the benefit of U.S. Provisional Patent Application No. 63/003,679, filed Apr. 1, 2020, entitled AUTONOMOUS SMART UNIVERSAL SANITIZATION IoT SYSTEM, of U.S. Provisional Patent Application No. 63/019,555, filed May 4, 2020, entitled AUTONOMOUS SMART UNIVERSAL , SANITIZATION IoT SYSTEM, and of U.S. Provisional Patent Application No. 63/076,414 filed Sep. 10, 2020, entitled AUTONOMOUS SANITIZATION MANAGEMENT CONTROL, AND DATA PLATFORM (ASMCDP), the entirety of each being respectively incorporated herein by reference.

### FIELD OF THE INVENTION

The present innovation is related to industrial sanitization, and more particularly to an autonomous sanitization management control, and data platform (ASMCDP), and still more particularly to an ASMCDP to manage and control large scale sanitization, globally and locally, across different industries—especially mobility and transportation, and even more particularly an ASMCDP to enable local and global sanitization data acquisition on-the-go, as well as, enabling data acquisition of related biological data.

### BACKGROUND OF THE INVENTION

Systematic sanitization is vital in all places for human health and safety, food production, as well as all sensitive industries, such as but not limited to healthcare, hospitals, pharmaceutical, space and aerospace, transportation, agricultural, entertainment, textile, clothing and fashion, homes, hotels and all other sensitive industries.

Existing sanitization methods are mainly dependent on humans to use sanitizing materials such as soaps and alcohols for personal use as well as cleaning of environments. This procedure is extremely labor-intensive, slow and highly prone to human errors while also failing to be systematic and barely manageable or traceable. This is especially evident in the case of pandemics, like COVID-19, in which it is almost impossible to cover all and every aspect of disinfection. Particular challenges include quick and frequent sanitization of objects and places that are constantly in contact with humans, such as but not limited to, public transportation like bus, taxi and airplane seats or public toilets after every usage.

The efficiency of UV light for destroying virus DNA has been demonstrated. The American Society of Heating, Refrigerating and Air-Conditioning Engineers (ASHRAE) recommend Ultraviolet Germicidal Irradiation as one strategy to address COVID-19 disease transmission. Ultraviolet Germicidal Irradiation (UVGI) is already commercially available for health services providers and may come in a portable or fixed form. Also available is a robotized version of an UVGI for hospitals that operates on a routine and is connected to wireless internet and powered by battery.

Thus, there is a need for systematic and autonomous sanitization across a number of industries, including but not limited to mobility and transportation, including cars, subways and airplanes, and public places such as but not limited to airports, hospitals, offices and public toilets. The present invention meets these, and other, needs.

### BRIEF SUMMARY OF THE INVENTION

In accordance with an aspect of the present invention, there is provided an autonomous sanitization management control, and data platform (ASMCDP) that addresses managing and controlling on-the-go or/and per-usage customized sanitization. For example, sanitizing the check-in kiosks in airports after each usage (per passenger) or enabling control and management of ambulances and/or taxis sanitization when they are moving to pick up their next passengers. One embodiment of the present invention may enable the collection of sanitization data for each autonomous sanitization such as the number of sanitizations, its efficiency, its speed, the location of sanitization, biologic-related data of the environment, etc.

In another aspect of the present invention, a fully automated intelligent system is configured to manage and control sanitization including sanitization data acquisition. Other advantages of present sanitization solution may include: a) easy to manage while eliminating human errors while also being non-labor-intensive; b) smart and customizable for different applications to manage and control sanitization remotely; c) access to sanitization data and related data such as biological data and environmental data at the place of sanitization; d) compatible with a biosensor configured to detect nanoparticles of COVID-19, other diseases or other targets of choice.

In another aspect, the present invention may be directed towards systems and methods of utilizing sanitization related telematics data to improve sanitization management, planning and operations. According to various embodiments, a sanitization management system is provided for capturing, storing, and analyzing telematics data to improve sanitization management operation. The autonomous sanitization management system may be used, for example, as a Standalone Autonomous Sanitization (SAS) unit or as a robot, to capture telematics data from the unit sensors and to analyze the captured telematics data.

The sanitization management system may also be configured to assess various aspects of sanitization performance, such as duration, location/place and UV dosage and pattern of sanitization. These analytical capabilities allow the sanitization management system to assist sanitization managing entities, or other entities, in analyzing local and global sanitization performance, disinfecting pathogens/viruses and maintenance costs, and improving sanitization planning and operation.

The ASMCDP enables an intelligent sanitization management, control, and data acquisition system relating to sanitization control technical fields. In one aspect of the invention, the ASMCDP may include setting up a Standalone Autonomous Sanitization (SAS) unit in every location of sanitization.

### BRIEF DESCRIPTION OF THE DRAWINGS

The accompanying drawings form a part of this specification and are to be read in conjunction therewith, wherein like reference numerals are employed to indicate like parts in the various views, and wherein:

FIG. 1 is a schematic view of an embodiment of an autonomous sanitization management control, and data platform (ASMCDP) in accordance with an aspect of the present invention;

FIG. 2 is a schematic view of an embodiment of a Standalone Autonomous Sanitization (SAS) unit configured for use within the ASMCDP shown in FIG. 1;

FIG. 3 is a schematic view of a system of SAS units operating in a master/slave configuration;

FIG. 4 is a schematic overview of the components and the communication routes between two SAS units in an ASMCDP ecosystem;

FIG. 5 is a structural schematic of an embodiment of a SAS UV sanitization unit;

FIGS. 6A-6C are representative examples of locations suitable for use with SAS units and an ASMCDP system in accordance with the present invention; and

FIG. 7 is a schematic view of an embodiment of an SAS mounted onto an aerial drone.

### DETAILED DESCRIPTION OF THE INVENTION

This summary is provided to introduce a selection of concepts in a simplified manner that is further described in the cases provided below. This summary is not intended to identify all key or essential features of the claimed subject matter, nor is it intended to be used as an aid in determination of the whole scope of the claimed subject matter.

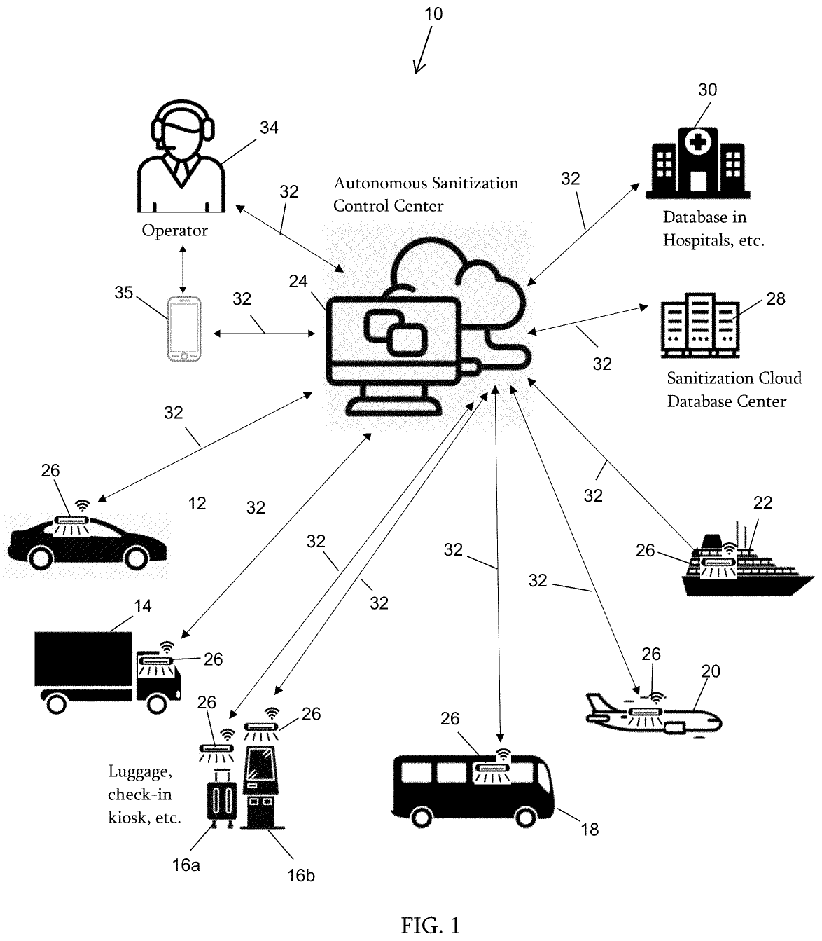

Turning now to the figures, FIG. 1 shows the schematics of an autonomous sanitization management control, and data platform (ASMCDP) 10 that may be used across a number of applications, including but not limited to the mobility/transportation sector, such as automobiles/taxis 12, mail/delivery services 14, airport terminal luggage check 16a/kiosks 16b, motorcoach/public transit/school buses 18, airplanes 20, cruise ships 22. ASMCDP 10 creates an ecosystem for autonomous sanitization including such features as “real-time autonomous sanitization data and information”, “remote autonomous sanitization control and management”, “bio-smart autonomous sanitization”, and “Al enabled sanitization”. Thus, ASMCDP 10 may enable for controlling, monitoring and managing autonomous sanitization remotely using an Autonomous Sanitization Control Center (ASCC) 24.

As shown in FIG. 1, Autonomous Sanitization Control Center (ASCC) 24 is in wireless communication with one or more respective Standalone Autonomous Sanitization (SAS) units 26 positioned so as to provide sanitization services to each of mobility/transportation units 12-22. ASCC 24 may further communicate with a sanitation cloud database center 28 and/or other client databases, such as a hospital database 30. ASMCDP 10 thus enables the observation and recording of sanitization communications data traffic 32 through sanitization data acquisition and information processing for global and local sanitization. The sanitization data can be stored in real-time in cloud database 28. An operator 34 may also communicate with ASCC 24 to verify, monitor and maintain ASMCDP 10, as necessary or desired. The sanitization data may also be communicated to a hand-held computing device, such as a tablet or smartphone 35 for operator 34 to observe and/or intervene in real-time manner.

In accordance with an aspect of the present invention, SAS units 26 may be considered the hardware building blocks of ASMCDP 10. All SAS units 26 are enabled for autonomous sanitization and may be connected to ASCC 24 by any form of wireless/wire coupling so as to enable transfer of any data or other information 32 collected by each SAS unit 26 to any cloud database 28 managed by ASCC 24. All SAS units 26 may also be controlled and managed by ASCC 24 automatically or through operator 34 input. In this manner, ASMCDP 10 enables controlling and monitoring of sanitization similar to what a traffic center does to monitor and control the traffic in a city. In other words, ASMCDP 10 offers a smart sanitization ecosystem by enabling all Internet of Things (IoT) sanitization devices (SAS units 26) to be connected to ASCC 24 and be managed by ASCC 24.

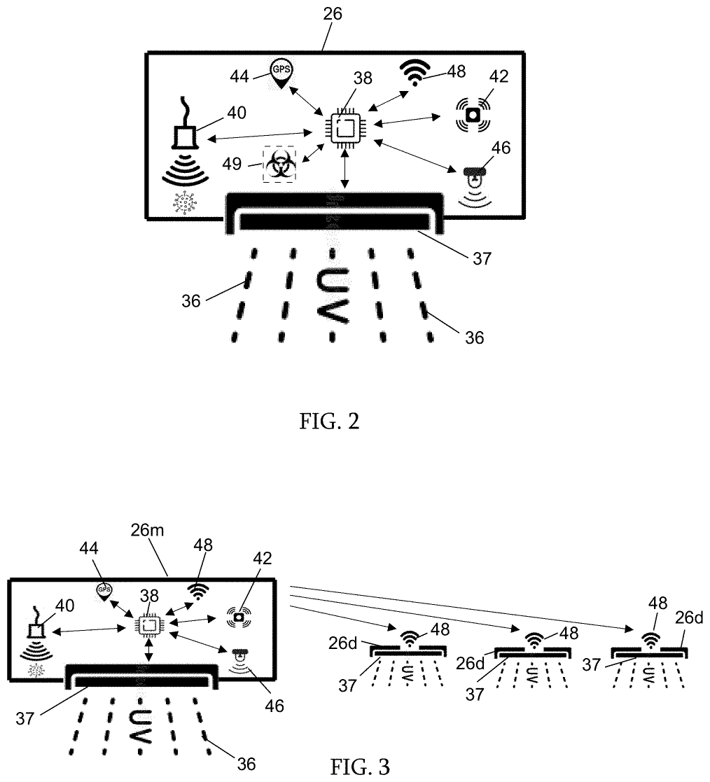

With reference to FIG. 2, an exemplary embodiment of a Standalone Autonomous Sanitization (SAS) unit 26 is shown. As represented by dashed lines 36, SAS unit 26 uses UV light 36 emitted from a UV light source 37 to sanitize a targeted environment. It should be noted that while SAS unit 26 is described as emitting UV light 36, additional or alternative wavelengths may be used in accordance with the teachings of the present invention. SAS unit 26 may include a microcontroller 38 in communication with various modalities so as to provide one or more features, such as but not limited to “Safety”, “Performance”, “Location”, “Connectivity” and “Bio-detection”. More specifically, SAS unit 26 may be equipped with one or more sensors, such as biosensor unit 40, humidity, temperature and motion detector unit 42, Global Positioning System (GPS) device 44 and UV light sensor 46 and a connectivity interface, such as wireless transceiver 48, input ports for extra sensor(s)/biosensor(s) 49, to effectuate such features, as will be discussed in greater details below. It should be noted that additional or alternative functionalities or features may be integrated into SAS unit 26. In one aspect of the present invention, the sensors are configured to sense movement, the presence of germs, pollution and/or viruses, air quality, water quality and other required parameters for a fully functional and efficient sanitization system. The sensors provide data for the system to perform any required actions, create data histories and facilitate data analysis.

Turning now to FIG. 3, in accordance with an aspect of the present invention, one SAS unit 26m may act as a Master unit (MSAS) and control a plurality of Dump (Slave) SAS (DSAS) units 26d. In accordance with this aspect, each DSAS unit 26d may be equipped with only a UV light source 37 and connectivity interface 48. Depending upon location or intended function respective DSAS units 26d may also include one or more specific sensors 40, 42, 44, 46 present on MSAS unit 26m. In this embodiment, the MSAS and DSAS units may enable the development of customized and/or cost-effective sanitization system solutions. It should be further noted that MSAS unit 26m and DSAS unit(s) 26d can use different connectivity architectures (wired/wireless) based on the use case. In one embodiment, MSAS and DSAS units 26m, 26d may be connected to Google NEST to be controlled by the NEST smart home platform so as to enable sanitization of households and commercial buildings.

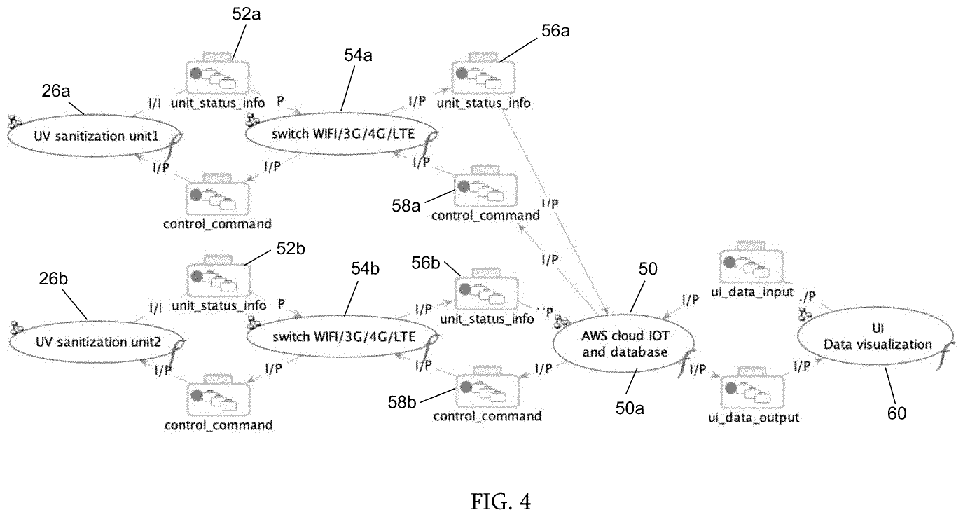

FIG. 4 shows a high-level overview of the components and the communication routes between two representative SAS units 26a, 26b in the ASMCDP ecosystem 10. FIG. 4 is meant to be an example for providing a general explanation of the components so as to clarify the general concept of a proposed model in accordance with the present invention, and is not meant to be in any way limiting thereto. Thus, FIG. 4 may represent the high-level view of multiple UV Sanitization SAS units 26 in interaction with an IoT cloud 50 (e.g., sanitation cloud database center 28) and may provide an application programming interface (API) providing a computing interface for a third party software and data platform, here for example AMAZON AWS IoT cloud as the Sanitization Cloud Database. SAS Units 26a, 26b transmit their status 52, 52b through available telecommunication channels 54a, 54b, including WIFI, LTE, 4G, etc., upon availability, and cache the data locally otherwise until a reliable channel is re-established. AWS IoT cloud 50 tracks all of the received statuses 56a, 56b and commands and stores them in the cloud database 50a. The proper telecommunication channel between ASCC 24 and the cloud components 50, 50a can be used. Available methods include Bluetooth and Wi-Fi (upon availability in short distances), 3G/4G/LTE (suitable for long ranges) and satellite internet. In one aspect of the present invention, the telecommunication channel 54a, 54b is chosen based on availability, cost, and necessary bandwidth.

AWS IoT cloud 50 may also transmit suitable adjustment settings, through control commands 58a, 58b, to each SAS unit 26a, 26b independently, depending upon each SAS unit's needs, in time and place. The frequency of the control command 58a, 58b may be adjustable, depending on the initial set-up and desired autonomy of each SAS unit 26, and is maintained by operator 34 or ASCC 24 (see FIG. 1). Operator 34 may control the settings of each SAS unit 26a, 26b, such as through user interface 60. Settings may be based upon a user desired scenario, such as for example, in a first, sporadic scenario (a) where microcontroller 38 is programmed to change commands independently of operator 34 or a second scenario (b) wherein user interface presents each SAS unit's status and command changes are set by operator 34. Thus, operator 34 can selectively adjust the autonomy of each SAS unit 26a, 26b, can switch between scenarios and may ultimately control one or both SAS units 26a, 26b, if desired.

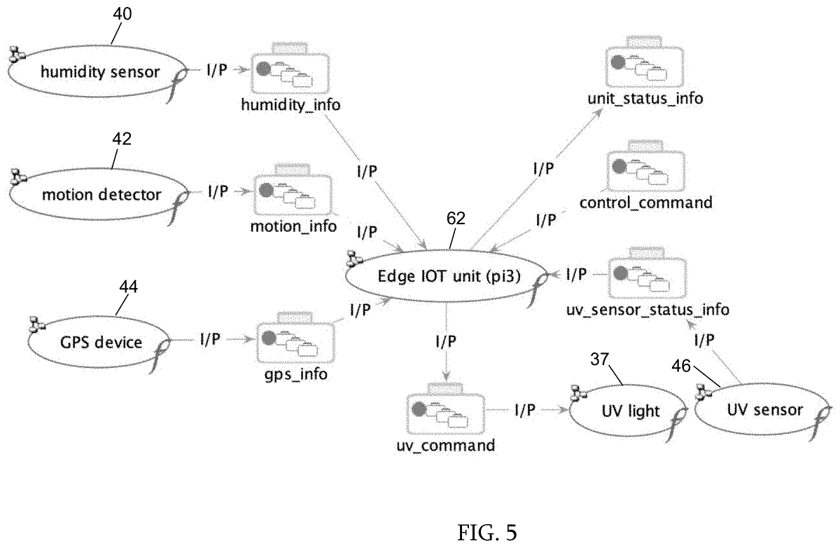

FIG. 5 shows an overview of the structure of a typical SAS unit 26, such as those shown and described above with regard to FIGS. 2 and 3. In accordance with an embodiment of the present invention, each SAS unit may include an edge IoT unit 62 responsible for gathering status information from all available sensors, such as but not limited to sensors 40, 42, 44, 46, 48 (see FIG. 2). Edge IoT unit 62 accepts control commands 58 from ASCC 24 or IoT database 50a and transmits back the status information 56 of all sensors 40, 42, 44, 46, 48 and the UV light source 37. Depending on the autonomy settings of the unit, edge IoT unit 62 may selectively fully control the entire system of SAS units 26, one or more selected SAS units 26, or allow operator 34 to control all or a portion of the SAS unit system.

With ASMCDP 10 properly set up with one or more SAS units 26 and associated sensors 40, 42, 44, 46, 48 (as desired), ASMCDP 100 provides constant, effective and safe autonomous sanitization processes. For example, humidity and temperature sensor 40 may monitor the environment such that UV light source 37 selectively outputs the correct UV light 36 dosage (i.e. exposure time and intensity of the UV light 36 under different weather conditions for higher performance by ensuring effective disinfection without using unnecessary extra dosage of UV light 36, preventing more electricity usage in vehicles by SAS and consuming extra life cycle of lamps for sake of less energy consumption and lowering the cost.

Motion detector and occupancy sensor 42 may monitor and transmit movement/occupancy status of the sensed to make sure appropriate safety measures are employed by UV source 37 (to turn off the UV sources or other lights in presence of humans/animals in the SAS UV range) since undue exposure to UV radiation is harmful for humans.

GPS device 44 reports the location of SAS unit 26 to ASCC 24 for optimal planning based on post-data analysis to record the location of sanitization in database 28, 30, the GPS may report the SAS location continuously, event based or on demand .

UV light sensor 46 monitors the UV radiation output by UV light source 37 and such that the minimally-required level (intensity and/or duration) of UV light 36 between wavelengths of about 100 to about 280 nanometers is outputted for safety and efficiency of the SAS unit 26. UV light sensor 46 may also calibrate the SAS unit 26 based on UV light intensity and adjust the UV dose, which is defined for SAS to target specific viruses/pathogens, as the UV light ages and degrades.

Each SAS unit 26 may also include extra slots for adding more sensor(s) and/or signaling system(s) 49, to SAS unit 26, and ultimately ASMCDP 10. For example, a biosensor 40 (see FIG. 2) may be added to an SAS unit 26 to create a “Bio-Smart SAS unit” 26s which may be programmed to sanitize the environment based on detection of a targeted virus or pathogen (biological data or bio-data). In one non-limiting example, a COVID-19 biosensor may be added to an SAS unit 26 whereby ASMCDP 10 monitors for and provides autonomous sanitization based on detection of the COVID-19 coronavirus.

Thus, ASMCDP 10 may enable global/local bio-data acquisition which enables “Bio-Smart Sanitization”, i.e., autonomous sanitization based on biosensors that can trace pathogens or viruses in the environment and initiate the SAS unit 26 to complete the required action for disinfection (UV dosage, i.e. emission of UV light 36 at the desired wavelength, intensity and duration. Bio-data acquisition may also enable the bio-data and associated information to be transferred and/or providing an API to a third party, such as hospitals, healthcare centers, governments or other system operators.

In accordance with an aspect of the present invention, all the sensors (i.e., one or more of sensors 40, 42, 44, 46, 48, 49) and UV light source 37 on SAS unit 26 are connected to a microprocessor and electronic board/microcontroller 38. Microprocessor and electronic board/microcontroller 38 may be connected to an IoT cloud-based ASCC system 24, such as through a wireless connectivity interface 48. ASCC 24 allows for control of UV light source 37 for sanitization whenever needed. Environmental data are collected by and reported from one or more sensors (40, 42, 44, 36, 49) to microcontroller 38 and are stored in the cloud database 28, 30 while ASMCDP 10 uses these data to manage sanitization in a real-time manner. In a sanitization case, microcontroller 38 activates UV light source 37 until UV light 36 UV exposure reaches an energy density of about 20,000 joules per square meter for a targeted surface under sanitization. Exposures of such density are sufficient for the disinfection of 99% of viruses, including coronavirus.

The IoT system of SAS units 26 and ASCC 24 enables the development of codes to run different patterns of UV light sanitization, while Al algorithms may learn in parallel for self-optimization. The data collected may include detection of viruses, the location and time stamp of UV sanitization, the amount of time spent by human(s) within the sensed area, human entrances into and exits out of the sensed area over a period of time, and, where applicable, patient biometrics.

The data collected from environments including traces of actions are stored for pattern recognition of sanitization including its human related behavior. Pattern recognition algorithms are performed to extract useful data, such as the potential risk of infection from any person in the room (visitors, care workers etc.), the location of the specific places that are more likely to be infected (such that UV light irradiation may be tailored to those places), cleaning time required to eliminate pathogens and UV sanitization interruption or incomplete because of human interruption and device failure/maintenance/diagnostics. The algorithms may then optimize an autonomous process/routine for the disinfection of the sensed area.

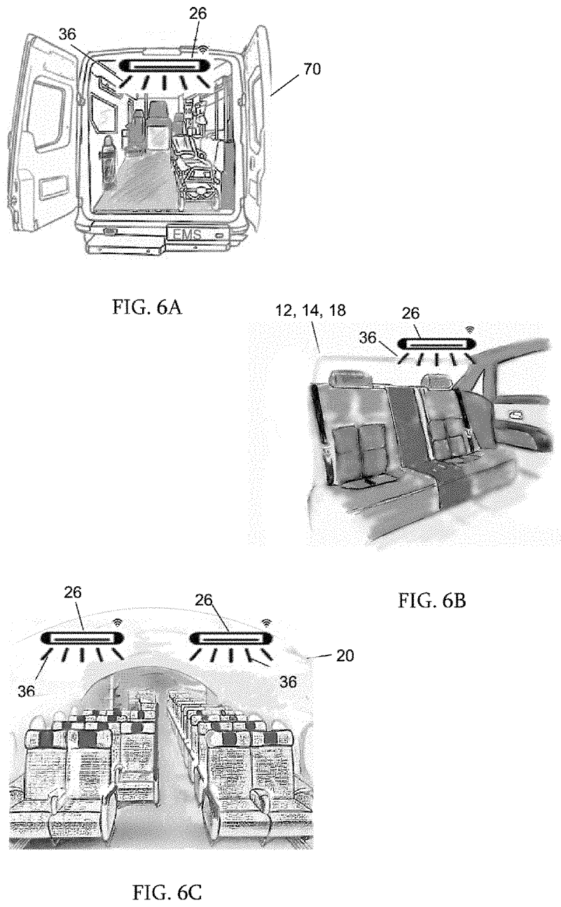

FIG. 6A shows an exemplary embodiment of an ASMCDP system 10A configured for ambulance 70 sanitization. At least one SAS unit 26 is operably connected within the ASMCDP 10A, such as through wireless 4G communication. SAS unit 26 may be monitored by an operator 34 in a hospital or other medical facility. In one aspect, SAS unit 26 may be installed in ambulance 70 as an Add-on (e.g., a “plug and play” unit) and may use ambulance power. In this embodiment, it has been found that motion sensor 42 communication with microcontroller 38 is critical and should have a data rate greater than about 0.2 second. Motion sensor 42, and thus ASMCDP 10A, can then recognize any movement within ambulance 70 and control emission of UV light 36 to ensure human safety. UV light sensor 46 is less critical and can have a lower communication rate of about 10 seconds or more. Firmware code identifies and alerts of any failed communication with sensors 42, 46. UV light source 37 may include a circuit board communication with microcontroller 38 for Off/On power control to UV light source 37 from the ambulance 70 batteries. In an alternative embodiment, UV light source 37 may include LEDs that are powered by dedicated batteries for convenience and flexibility.

FIG. 6B demonstrates an exemplary embodiment of an SAS unit 26 within ASMCDP 10B a taxi 12 or other passenger automobile, such as truck 14 or bus 18 (see FIG. 1). In accordance with an aspect of this embodiment, motion and occupancy sensor 42 prevents powering of UV light source 37 while a passenger is in the vehicle so as to eliminate possible UV exposure to the passenger. GPS sensor 44 enables identifying the location and time where vehicle 12, 14, 18 sanitization takes place. Each vehicle can thus be sanitized before each passenger enters the vehicle between runs. ASMCDP 10B sanitization can also be adapted through data analysis for health and safety insurance for each passenger.

FIG. 6C shows an exemplary embodiment of a plurality of SAS units 26 within an ASMCDP 10C configured for airplane 20 cabin sanitization. Each motion sensor 42 prevents powering of UV light source 37 while crew and passengers are in the airplane cabin. The sanitization procedure of ASMCDP 10C can be adapted to the policies of each individual aviation company and the data collected can be stored and analyzed for health and safety insurance. UV light sensor 46 can monitor exposure to ensure sufficient UV light has irradiated the cabin for safe disinfection. Biosensors 49 may also be added to the system to target any specific biologic agent under target.

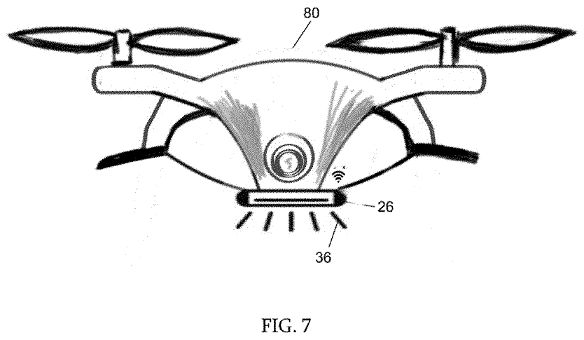

FIG. 7 illustrates an exemplary embodiment of SAS unit 26 mounted onto an unmanned aerial vehicle (UAV) or drone 80 to create ASMCDP 10D which may be able to manage and control sanitization in any location, even on a routine basis. In one aspect of the present invention, drone 80 may use GPS data received from GPS sensor 44 to track the location(s) which need(s) to be sanitized using UV light source 37 mounted on drone 80. These locations may be defined by operator 34 or ASCC 24. ASMCDP 10D may provide for a programmed sanitization procedure that defines where drone 80 needs to sweep so that UV light 36 can sanitize that defined path or track. By way of example and without limitation thereto, ASMCDP 10D may be used for airport sanitization and can be programmed based on the interior/exterior map of an airport.

In one aspect of this embodiment, the distance of drone 80 from the targeted surface can be adjusted so that drone 80 is at an optimal distance from the surface for higher intensity of UV light 36 exposure. The sanitation data can then be stored and analyzed for health and safety measures, insurances and policies. In another aspect drone 80 may be equipped with biosensor 40 if needed/desired. ASMCDP 10D, and thus drone 80 has IoT cloud connectivity via interface 48 for real-time monitoring and management of ASMCDP 10D.

From the above descriptions, it should be understood by those skilled in the art that ASMCDP 10 enables systematic management and control of disinfecting/sanitizing of any sector of the public transportation and mobility industry, such as but not limited to disinfecting luggage, aircrafts, trains, subways/metros/tubes, buses and taxis by using SAS units to disinfect and sanitize the seats and common areas after each usage. In one aspect, sanitization takes place in the absence of human intervention. Sensors can detect once a vehicle is empty and the ASMCDP system can then disinfect all seats and common areas through irradiation via UV light. Sanitization data, bio-data and any other relevant data is then stored in a database for use in such activities such as sanitization management of public transportations, for policy making and for medical planning and research.

ASMCDP 10 may also offer systematic management and control of sanitization with one or more of the following advantages: a) a complete and quick automated solution, both locally and globally, b) is easy to manage, is not labor-intensive and eliminates human error, c) is smart and customizable for different applications, d) provides traceable data and algorithms for a fully customizable sanitization management, e) provides guidance for policy makers in passing new laws/regulations, f) remote troubleshooting and maintenance of SAS units in Sanitization ecosystem enabling efficient and quick customer support with less interruption in systematic sanitization and g) reprogramming a group of SAS units globally/locally to disinfect a new virus or biosafety threat in urgent cases in a short period, for example a few hours.

Although the invention has been described with reference to preferred embodiments thereof, it is understood that various modifications may be made thereto without departing from the full spirit and scope of the invention as defined by the claims which follow.

What is claimed is:

1. An autonomous sanitization management, control and data platform (ASMCDP) for sanitizing an area, the ASMCDP comprising:
a) an autonomous sanitization control center (ASCC); and
b) one or more standalone autonomous sanitization (SAS) units, wherein each of said one or more SAS units comprises:
i) at least one sensor configured to collect sensor data;
ii) a microcontroller communicatively coupled to said at least one sensor;
iii) internal software/firmware configured to be executed by said microcontroller to enable said each of said one or more SAS units to work autonomously as said microcontroller adjusts functionality of said each of said one or more SAS units with respect to said collected sensor data;
iv) a light source coupled to said microcontroller, wherein power to said light source is selectively controlled by said microcontroller in response to a triggering signal emitted by said at least one sensor whereby when said light source is powered on said light source emits sanitizing radiation; and
v) an Internet-of-Things (IoT) communication unit communicatively coupled to said microcontroller and said ASCC.

2. The autonomous sanitization management, control and data platform of claim 1, wherein said at least one sensor is a humidity and temperature sensor unit, a motion detector, a global positioning system (GPS) device, a light sensor, a biosensor, or combinations thereof,

3. The autonomous sanitization management, control and data platform of claim 1, wherein said light source emits ultraviolet (UV) radiation and/or other light radiation.

4. The autonomous sanitization management, control and data platform of claim 3, wherein said light source emits ultraviolet (UV) radiation until said radiation reaches a predetermined energy density based on a task assigned to said one or more SAS units.

5. The autonomous sanitization management, control and data platform of claim 1, wherein each of said standalone autonomous sanitization (SAS) units further comprises a wireless transceiver.

6. An autonomous sanitization management, control and data platform comprising:
a) a vehicle having an interior passenger compartment;
b) one or more standalone autonomous sanitization (SAS) units mounted within said interior passenger compartment; and
c) an autonomous sanitization control center (ASCC) remotely located from said vehicle;
wherein each of said one or more SAS units comprises:
i) at least one sensor configured to collect sensor data;
ii) a microcontroller communicatively coupled to said at least one sensor;
iii) internal software/firmware configured to be executed by said microcontroller to enable said each of said one or more SAS units to work autonomously as said microcontroller adjusts functionality of said each of said one or more SAS units with respect to said collected sensor data;
iv) a light source coupled to said microcontroller, wherein power to said light source is selectively controlled by said microcontroller in response to a triggering signal emitted by said at least one sensor whereby when said light source is powered on said light source emits sanitizing radiation; and
v) an Internet-of-Things (IoT) communication unit communicatively coupled to said microcontroller and said ASCC.

7. The autonomous sanitization management, control and data platform of claim 6, wherein said at least one sensor is a humidity and temperature sensor unit, a motion detector, a global positioning system (GPS) device, a light sensor, a biosensor, or combinations thereof,

8. The autonomous sanitization management, control and data platform of claim 6, wherein said light source emits ultraviolet (UV) radiation and/or other light radiation.

9. The autonomous sanitization management, control and data platform of claim 8, wherein said light source emits ultraviolet (UV) radiation until said radiation reaches a predefined energy density.

10. The autonomous sanitization management, control and data platform of claim 6, wherein each of said standalone autonomous sanitization (SAS) units further comprises a wireless transceiver.

11. The autonomous sanitization management, control and data platform of claim 7, wherein said light source is powered on only after said motion detector indicates no human and/or animal within said interior passenger compartment.

12. The autonomous sanitization management, control and data platform of claim 7, wherein said humidity and temperature sensor unit and said light sensor regulate output intensity of said light source depending upon sensed environmental conditions within said interior passenger compartment.

13. The autonomous sanitization management, control and data platform of claim 7, further comprising a database communicatively coupled to said ASCC whereby data generated by said one or more SAS units is stored within said database.

14. The autonomous sanitization management, control and data platform of claim 13, wherein said GPS device generates a data log of sanitization data including a location of said one or more SAS units and a time when said light source is powered on, wherein said data log is communicated to and stored within said database.

15. The autonomous sanitization management, control and data platform of claim 14, wherein said ASCC is programmed to include one or both of an artificial intelligence algorithm and a pattern recognition algorithm.

16. The autonomous sanitization management, control and data platform of claim 13, wherein said ASCC is configured to communicate with an operator whereby said one or more SAS units are configured to receive operating instructions from the operator.

17. The autonomous sanitization management, control and data platform of claim 16, further comprising a hand-held computing device, and wherein said ASCC communicates said data to said hand-held computing device.

18. An autonomous sanitization management, control and data platform comprising:
a) an unmanned aerial vehicle (UAV);
b) one or more standalone autonomous sanitization (SAS) units mounted on said UAV; and
c) an autonomous sanitization control center (ASCC) remotely located from said UAV;
wherein each of said one or more SAS units comprises:
i) at least one sensor configured to collect sensor data;
ii) a microcontroller communicatively coupled to said at least one sensor;
iii) internal software/firmware configured to be executed by said microcontroller to enable said each of said one or more SAS units to work autonomously as said microcontroller adjusts functionality of said each of said one or more SAS units with respect to said collected sensor data;
iv) a light source coupled to said microcontroller, wherein power to said light source is selectively controlled by said microcontroller in response to a triggering signal emitted by said at least one sensor whereby when said light source is powered on said light source emits sanitizing radiation; and
v) an Internet-of-Things (IoT) communication unit communicatively coupled to said microcontroller and said ASCC.

19. The autonomous sanitization management, control and data platform of claim 18, wherein said at least one sensor is a humidity and temperature sensor unit, a motion detector, a global positioning system (GPS) device, a light sensor, a biosensor, or combinations thereof,

20. The autonomous sanitization management, control and data platform of claim 18, wherein said light source emits ultraviolet (UV) radiation until said radiation reaches a predetermined energy density.
